(19) United States
(12) Patent Application Publication
(10) Pub. No.: US 2016/0324471 A1
(43) Pub. Date: Nov. 10, 2016

(54) WARNING DEVICE AGAINST CARDIAC ARREST CAUSED BY ARRHYTHMIA AND SLEEPING MONITORING BAND

(71) Applicant: JOO HYUNG LEE, Anaheim, CA (US)

(72) Inventor: JOO HYUNG LEE, Anaheim, CA (US)

(21) Appl. No.: 14/708,112

(22) Filed: May 8, 2015

(51) Int. Cl. A61B 5/00; A61B 5/026

(52) U.S. Cl. CPC A61B 5/6824; A61B 5/0261; A61B 5/746; A61B 5/74; A61B 5/7405; A61B 5/4806

(57) ABSTRACT

A wrist band shaped device which can accurately warn when there is something wrong with heart by reducing interference of noise and by easily detecting heart condition in everyday life.

### FIELD OF INVENTION

This invention is about a device that alarms cardiac arrest or dangerous situations by transmitting an alarm by wireless when a change in blood flow by detecting a change in blood flow via a device which calculates and compares blood flow with bio signal acquired through preprocessor. This invention is composed of a device which detects blood flow in wrist when heart stops and transmits it by wire or wireless, a device which alarms, sensor part which consists of means to send visible ray and infrared ray and to receive optical sensor and preprocessor part which filters and amplifies noises from sensor part output signals

### BACKGROUND OF INVENTION

Arrhythmia is highly liable to develop into cardiac arrest. If cardiac arrest occurs while a patient sleeps, it may cause death. However, it is very difficult to cope with cardiac arrest caused by arrhythmia during everyday life.

An effective way to treat cardiac arrest may be with a defibrillator. A defibrillator is a medical device that delivers an electric current, or “shock” to the chest of a cardiac arrest victim, which delivers that electric current to the victim's heart. The shock can interrupt the erratic electrical pulse of ventricular fibrillation of the heart during the cardiac arrest, which can give the victim's heart a chance to begin beating at its normal rhythm.

During the first several minutes after a person suffers a cardiac arrest, every minute saved before defibrillation may mean about a 10 percent increase in relative survival rate. Therefore, as time is critical, there has been an increase in the availability of automated external defibrillators, or AEDs. AEDs are portable medical devices designed primarily for use by “first responders” (first to arrive at the scene of a medical emergency), such as police and fire departments. An AED generally has a built-in computer that evaluates a victim's heat rhythm to determine whether a shock is required. AEDs are also relatively easy to use as they typically include voice instructions and screen messages to assist in its operation, and thus, can be used by an average citizen with little or no training.

Therefore an optimum device which is very simple and convenient to use in everyday life is necessary to effectively alarm cardiac arrest caused by arrhythmia and an alarm should be designed in such a manner that can effectively warn situations relating to cardiac arrest in everyday life.

This cardiac arrest alarm is system that detects whether blood flow is normal and oxygen saturation in blood in phenomena which vary depending on heart pulsation and amount of oxygen which combines with hemoglobin when infrared ray coming from radiating part and visible ray with specific wavelength reaches a part which receives rays passing through skin and bio tissues and if blood flow or oxygen saturation is below standard and warns by sending a signal to an alarm or devices designated.

### SUMMARY OF INVENTION

The purpose of this wrist band shaped invention is to easily detect the condition of heart in daily life and to accurately warn when there is something wrong with heart by reducing noise.

### BRIEF DESCRIPTION OF DRAWINGS

FIG. 1 illustrates a sensor circuit diagram in parts which send and receive infrared ray in wrist attached band.

FIG. 2 illustrates a block diagram of sensor, preprocessor part, first comparator and alarm transmitting part.

FIG. 3 illustrates a alarm wireless receiving part and warning speaker.

FIG. 4 illustrates a diagram about classification of pulsation.

### DETAILED DESCRIPTION OF EMBODIMENTS

Accurate measurement of blood flow is required to achieve the purpose of this invention. Wrist connecting side 10 and parts that send rays 20, 21 require means to closely attach in order to remove outside noise. This invention should be shaped like a decoration such as a ring for convenience in everyday life and parts including sensors should be miniaturized.

Existing devices to alarm heart attack are almost impossible to use in everyday life because they were designed to be worn in fingers or toes. The detector of this invention can measure precisely like medical instrument and it is convenient to wear and carry.

Therefore this invention is an effective device that can minimize a danger by sending an alarm a place designated or set by a user through aforementioned detector in case of sudden death caused by arrhythmia which a patient suffers while sleeping.

This invention will be examined by referring to drawings. If it is deemed that detailed description of technologies or compositions makes summary of this invention unclear, the detailed description will be left out.

Terms that will be described later and defined considering functions in this invention are subject to change by intention or practice of user or operator and the definitions should be made based on contents in this statement that describes this invention.

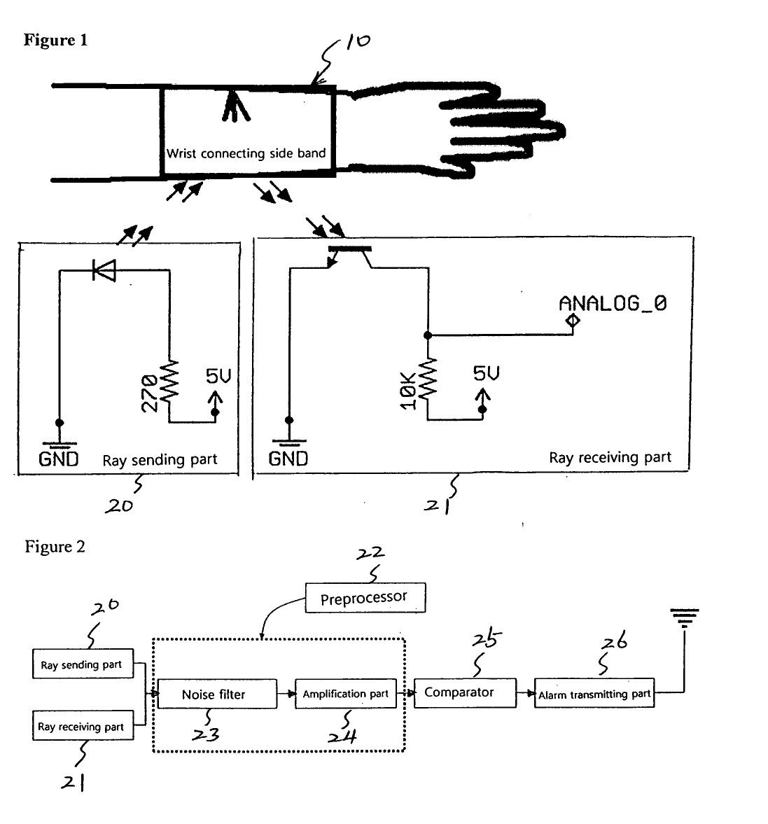

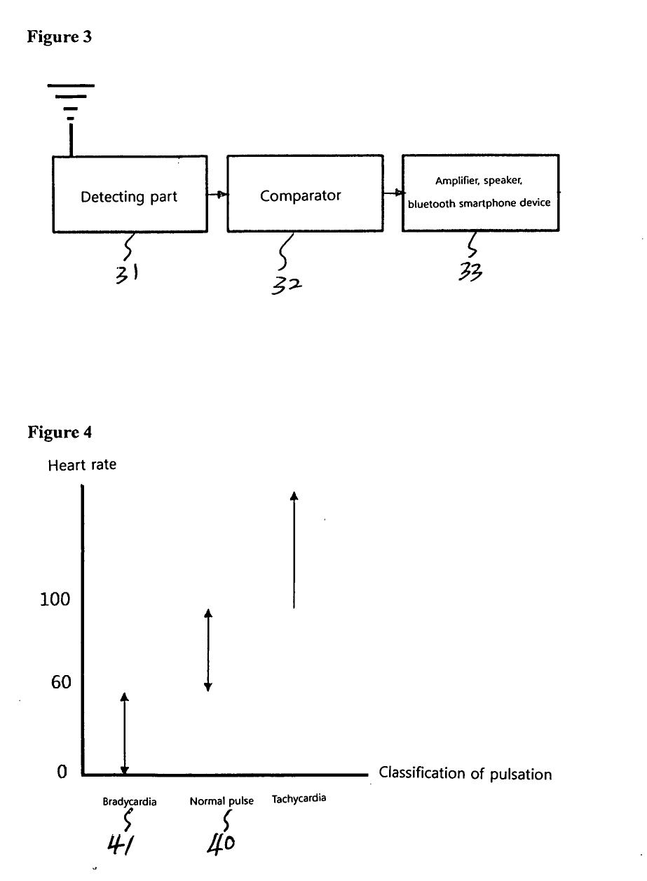

Visible ray and infrared ray that are sent to radiating part 20 at wrist connecting side band 10 in FIG. 1 which are received in a part 21 and FIG. 2 is composed of preprocessor 22 which is made up of noise filter 23 and amplification part 24 and noise is removed at noise filter and heart rate depending on blood flow from bio signal which is extracted via amplification part 24 is calculated and when it becomes bradycardia 41 which blood flow drops remarkably at first comparative part 25 compared with normal pulse 40 in FIG. 4 and it is sent to detecting part 31 in FIG. 3 from alarm transmitting part 26 in FIG. 2 and second comparative part 32 determines whether it is warning signal and an alarm is sent to amplifier, speaker and blue tooth smart phone device 33.

Alarm transmitting part 26 in FIG. 2 should be wireless one but can be wire one if necessary. Making signal digital transmission through encoding to reduce errors helps minimize error range.

Moreover, this warning device will be using as a sleeping monitoring band during sleep.

While certain exemplary techniques have been described and shown herein using various methods and systems, it should be understood by those skilled in the art that various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter not be limited to the particular examples disclosed, but that such claimed subject matter also may include all implementations falling within the scope of the appended claims, and equivalents thereof.

1. A band shaped wireless alarm device for detecting cardiac arrest.

2. The device recited in claim 1 wherein,
said device can be used as a sleeping monitoring band during sleep.

3. The warning device for detecting cardiac arrest, comprising of:
the ray sending part which sends a ray;
the receiving part which receives a ray that detects blood flow in wrist when a heart stops;
the preprocessor which filters and amplifies noises out of sensor output signals;
the comparator which calculates and compares blood flow via bio signals obtained through preprocessor; and
the wireless alarm device which warns dangerous situations by transmitting an alarm by wireless when there is something wrong.

4. A device as recited in claim 3, wherein the preprogrammed information comprises previous cardiac rhythm data of a person of the cardiac monitoring device.
